(19) United States
(12) Patent Application Publication
(10) Pub. No.: US 2007/0286906 A1
(43) Pub. Date: Dec. 13, 2007

(54) DIHYDROBENZOQUINONE COMPOUNDS

(75) Inventors: Weihan Zhang, Pudong (CN); Weiguo Su, Shanghai (CN); Yumin Cui, Shanghai (CN); Yongxin Ren, Shanghai (CN); Xiaoqiang Yan, Shanghai (CN); Jifeng Duan, Shanghai (CN)

Correspondence Address: OCCHIUTI ROHLICEK & TSAO, LLP, 10 FAWCETT STREET, CAMBRIDGE, MA, 02138, US

(73) Assignee: Hutchison MediPharma Enterprises Limited, Nassau (BS)

(21) Appl. No.: 11/741,247

(22) Filed: Apr. 27, 2007

Related U.S. Application Data
(60) Provisional application No. 60/796,164, filed on Apr. 28, 2006.

(51) Int. Cl. A61K 31/195; A61K 31/337; A61K 31/403; A61K 31/44; A61K 31/505; A61K 33/24; A61K 38/16; A61P 35/00; C07C 323/32; C07K 5/037

(52) U.S. Cl. 424/649; 514/274; 514/283; 514/411; 514/449; 514/562; 514/8; 530/331; 560/17

(57) ABSTRACT

Dihydrobenzoquinone compounds of the following formula: 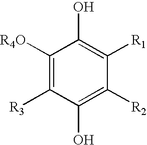
wherein R₁, R₂, R₃, and R₄ are defined herein. Also disclosed is a method of treating cancer with one of the dihydrobenzoquinone compounds.

### CROSS-REFERENCE RELATED APPLICATIONS

This application claims the benefit of U.S. Provisional Application Ser. No. 60/796,164, “DIHYDROBENZOQUINONE COMPOUNDS”, filed Apr. 28, 2006, which is incorporated herein by reference.

### BACKGROUND

Cancer, a leading fatal disease, features an abnormal mass of malignant tissue resulting from excessive cell division. Cancer cells proliferate in defiance of restraints on cell growth, and invade and colonize territories reserved for other cells.

Modes of cancer therapy include chemotherapy, surgery, radiation, and combinations of these treatments. In chemotherapy, a cancer patient is treated with one or more compounds that inhibit cancer cell growth. There is a need to develop more efficacious chemotherapeutic agents that can be used alone or in combination with other chemotherapeutic agents or radiation therapy.

### SUMMARY

This invention is based on an unexpected finding that certain dihydrobenzoquinone compounds exhibit inhibitory effects on cancer cell growth.

One aspect of this invention is a dihydrobenzoquinone compound of formula (I): 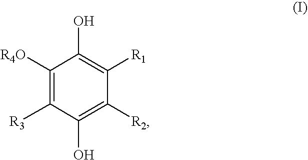
wherein R₁ is C₁₋₂₀ alkyl, C₂₋₂₀ alkenyl, C₂₋₂₀ alkynyl, or aryl; one of R₂ and R₃ is XRₐ and the other is H, C₁₋₆ alkyl, C₂₋₆ alkenyl, C₂₋₆ alkynyl, C₃₋₁₂ cycloalkyl, C₁₋₁₂ heterocycloalkyl, aryl, heteroaryl, or YR_{b}; in which each of X and Y, independently, is O, S, Se, or NR′, and each of Rₐ and R_{b}, independently, is H, C₁₋₆ alkyl, C₂₋₆ alkenyl, C₂₋₆ alkynyl, C₃₋₁₂ cycloalkyl, C₁₋₁₂ heterocycloalkyl, aryl, heteroaryl, C(O)R′, S(O)₂R′, an amino acid moiety, or an oligopeptide moiety; R′ being H, C₁₋₆ alkyl, C₂₋₆ alkenyl, C₂₋₆ alkynyl, C₃₋₁₂ cycloalkyl, C₁₋₁₂ heterocycloalkyl, aryl, or heteroaryl; and R₄ is H, C₁₋₆ alkyl, C₂₋₆ alkenyl, C₂₋₆ alkynyl, C₃₋₁₂ cycloalkyl, C₁₋₁₂ heterocycloalkyl, aryl, or heteroaryl.

Referring to formula I, one subset of the compounds feature that R₄ is (CH₂)₉CH═CH(CH₂)₅CH₃ or (CH₂)₁₆CH₃. Another subset of the compounds feature that R₄ is CH₃. Still another subset of the compounds feature that one of R₂ and R₃ is XRₐ and the other is H. Yet another subset of the compounds feature that X is S, and Rₐ is 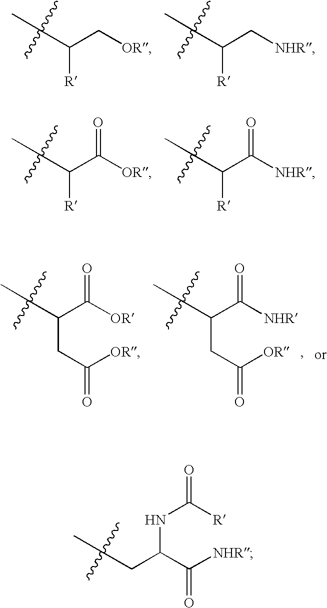
wherein R′ and R″ is H, C₁₋₆ alkyl, C₂₋₆ alkenyl, C₂₋₆ alkynyl, C₃₋₁₂ cycloalkyl, C₁₋₁₂ heterocycloalkyl, aryl, or heteroaryl, or Rₐ is 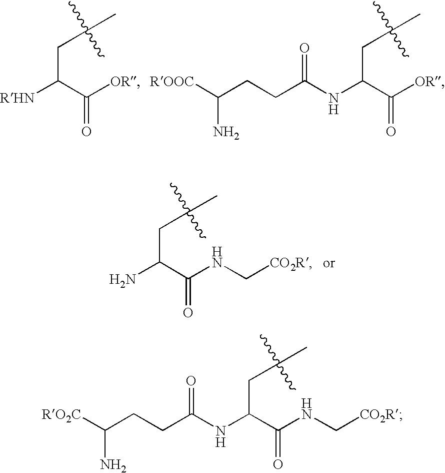
wherein R′ and R″ is H, C₁₋₆ alkyl, C₂₋₆ alkenyl, C₂₋₆ alkynyl, C₃₋₁₂ cycloalkyl, C₁₋₁₂ heterocycloalkyl, aryl, or heteroaryl.

The dihydrobenzoquinone compounds of this invention may contain one or more asymmetric centers. Thus, they occur as racemates and racemic mixtures, single enantiomers, individual diastereomers, diastereomeric mixtures, or cis- or trans-isomeric forms. All such isomeric forms are contemplated.

The term “alkyl,” unless stated otherwise, refers to a straight or branched hydrocarbon containing 1-20 carbon atoms. Examples of alkyl groups include, but are not limited to, methyl, ethyl, n-propyl, i-propyl, n-butyl, i-butyl, and t-butyl.

The term “alkenyl” refers to a straight or branched hydrocarbon having one or more carbon-carbon double bonds. The term “alkynyl” refers to a straight or branched hydrocarbon having one or more carbon-carbon triple bonds. The alkenyl and alkynyl, unless stated otherwise, contain 1-20 carbon atoms.

The term “aryl”, unless stated otherwise, refers to a 6-carbon monocyclic, 10-carbon bicyclic, 14-carbon tricyclic aromatic ring system wherein each ring may have 1 to 4 substituents. Examples of aryl groups include, but are not limited to, phenyl, naphthyl, and anthracenyl.

The term “cycloalkyl,” unless stated otherwise, refers to a saturated and partially unsaturated cyclic hydrocarbon group having 3 to 12 carbon atoms. Examples of cyclyl groups include, but are not limited to, cyclopropyl, cyclobutyl, cyclopentyl, cyclopentenyl, cyclohexyl, cyclohexenyl, cycloheptyl, and cyclooctyl.

The term “heterocycloalkyl,” unless stated otherwise, refers to a nonaromatic 3-8 membered monocyclic, 8-12 membered bicyclic, or 11-14 membered tricyclic ring system having one or more heteroatoms (such as O, N, or S) and 3 or 12 carbon atoms. Examples of heterocyclyl groups include, but are not limited to, piperazinyl, pyrrolidinyl, dioxanyl, morpholinyl, and tetrahydrofuranyl.

The term “heteraryl” refers to an aromatic 5-8 membered monocyclic, 8-12 membered bicyclic, or 11-14 membered tricyclic ring system having one or more heteroatoms (such as O, N, or S). Examples of heteroaryl groups include pyridyl, furyl, imidazolyl, benzimidazolyl, pyrimidinyl, thienyl, quinolinyl, indolyl, and thiazolyl.

The term “amino acid moiety” refers to a hydrocarbon moiety containing both at least one amino group and at least one carboxyl group. Examples include, but are not limited to, moieties derived from naturally occurring α-amino acids. The term “oligopeptide moiety” refers to a group composed of 2-10 amino acids linked to one another via an amide bond.

Alkyl, alkenyl, alkynyl, cycloalkyl, heterocycloalkyl, aryl, heteroaryl, an amino acid moiety, and an oligopeptide moiety can be either substituted or unsubstituted. For examples, these moieties can be substituted with groups containing zero to six heteroatoms selected from halogen, oxygen, sulfur, and nitrogen. Possible substituents on alkenyl, alkynyl, cycloalkyl, heterocycloalkyl, aryl, and heteroaryl, include alkyl, alkenyl, alkynyl, cycloalkyl, cycloalkenyl, alkoxy, aryl, aryloxy, heteroaryl, heteroaryloxy, amino, alkylamino, dialkylamino, arylamino, diarylamino, hydroxyl, halogen, thio, alkylthio, arylthio, alkylsulfonyl, arylsulfonyl, acylamino, aminoacyl, amidino, guanidino, ureido, cyano, nitro, acyl, acyloxy, carboxyl, and carboxylic ester. Possible substituents on alkyl include all of the above-recited substituents except alkyl.

Set forth below are a number of dihydrobenzoquinone compounds of this invention:

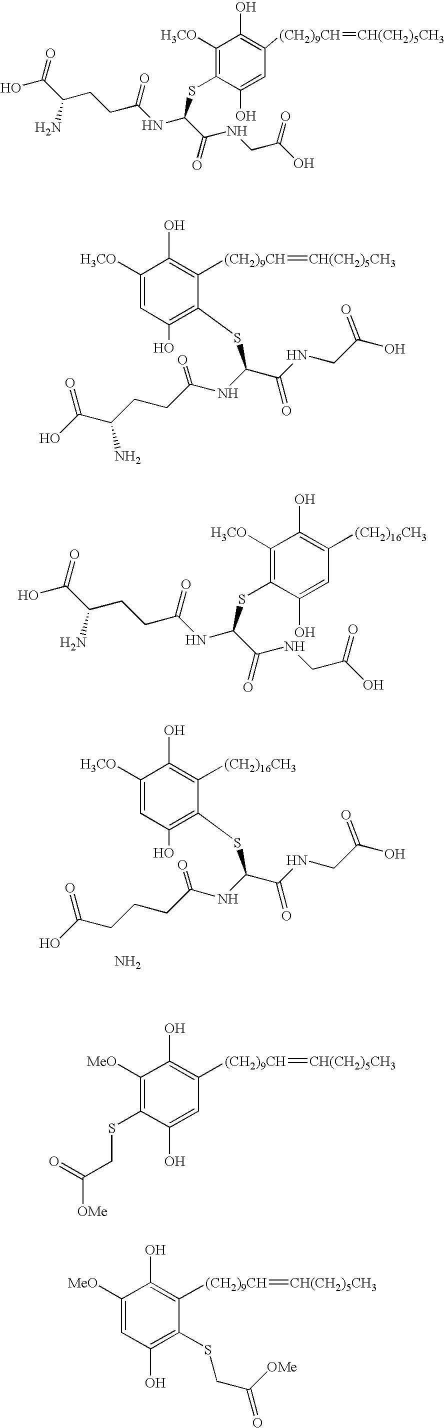

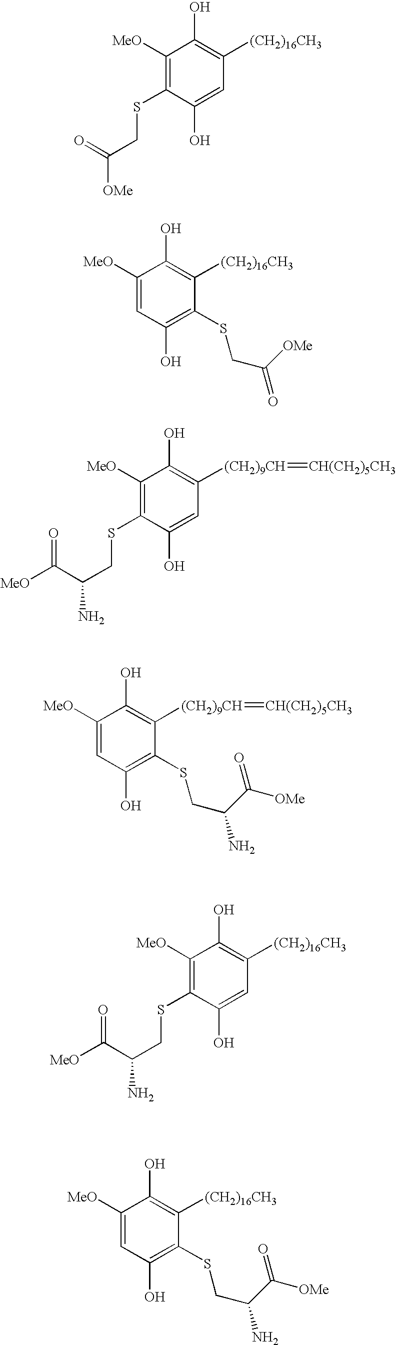

Another aspect of this invention is a process for preparing dihydrobnzoquinone compounds having formula (II): 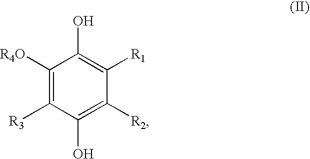
wherein R₁ is C₁₋₂₀ alkyl, C₂₋₂₀ alkenyl, C₂₋₂₀ alkynyl, or aryl; one of R₂ and R₃ is XRₐ and the other is H; in which X is O, S, Se, or NR′; and Rₐ is H, C₁₋₆ alkyl, C₂₋₆ alkenyl, C₂₋₆ alkynyl, C₃₋₁₂ cycloalkyl, C₁₋₁₂ heterocycloalkyl, aryl, heteroaryl, C(O)R′, S(O)₂R′, an amino acid moiety, or an oligopeptide moiety; R′ being H, C₁₋₆ alkyl, C₂₋₆ alkenyl, C₂₋₆ alkynyl, C₃₋₁₂ cycloalkyl, C₁₋₁₂ heterocycloalkyl, aryl, or heteroaryl; and R₄ is H, C₁₋₆ alkyl, C₂₋₆ alkenyl, C₂₋₆ alkynyl, C₃₋₁₂ cycloalkyl, C₁₋₁₂ heterocycloalkyl, aryl, or heteroaryl. The process includes reacting HXRₐ, in which X and Rₐ are defined above, with a compound of formula (III): 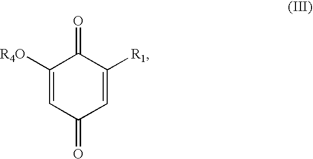
R₁ and R₄ are defined above.

Still another aspect of this invention is a method of treating cancer (e.g., oesophagus carcinoma, head and neck carcinoma, gastric carcinoma, lung carcinoma, or colon carcinoma). The method includes administering to a subject in need of the treatment an effective amount of one or more of the dihydrobenzoquinone compounds of this invention. In this method, one, two, or even more additional chemotherapeutic agents (other than a dihydrobenzoquinone compound) may be co-administered or the subject may be co-treated with radiation therapy. Examples of additional chemotherapeutic agents include, but are not limited to, cisplatin, mitomycin C, bleomycin, topotecan, irinotecan, gemcitabine, docetaxel, paclitaxel, podophyllotoxin, vincristin, plicamycin, daunorubicin, dactinomycin, adriamycin, 5-fluorouracil, hormones, hormone antagonists, and cytokines (e.g., interleukin-2 and transforming growth factor β). Radiation therapy refers to treatment with either ionizing radiation or non-ionizing radiation.

Also within the scope of this invention is a composition containing one or more the dihydrobenzoquinone compounds, optionally one or more additional chemotherapeutic agents, and a pharmaceutically acceptable carrier, as well as the use of such a composition for the manufacture of a medicament used in cancer treatment.

Details of several embodiments of the invention are set forth in the description below. Other features, objects, and advantages of the invention will be apparent from the description, and also from the claims

### DETAILED DESCRIPTION

The dihydrobenzoquinone compounds of this invention can be prepared from commercially available reagents or naturally-occurring compounds via conventional chemical transformations, for example, those described in R. Larock, Comprehensive Organic Transformations, VCH Publishers (1989); T. W. Green and P. G. M. Wuts, Protective Groups in Organic Synthesis, 3^{rd} Ed., John wile and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

As illustrated in Scheme 1 below, certain dihydrobenzoquinone compounds of this invention can be prepared by reacting a nucleophilic agent (H—X—Rₐ) with Irisquinone A (IqA) or Irisquinone B (IqB). IqA and IqB are naturally occurring compounds isolated from the seed coating of Iris pallassi Fisch. var chinensis Fisch. and the seed oil of Iris pseudacorus L. See U.S. patent application Ser. No. 11/156,210.

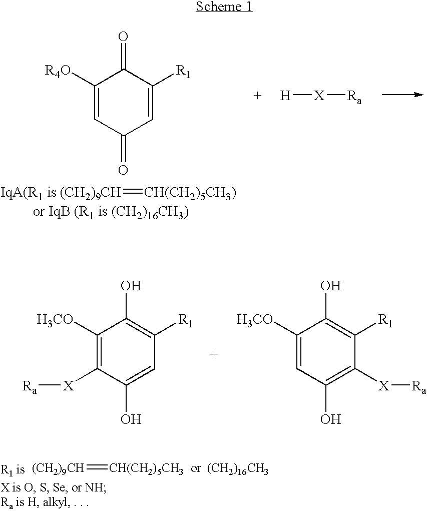

The dihydrobenzoquinone compounds of this invention inhibit growth of tumor cells. Thus, this invention relates to a method of treating cancer by administering to a subject in need thereof an effective amount of one or more of these compounds. The term “an effective amount” refers to the amount of a dihydrobenzoquinone compound that is required to confer the intended therapeutic effect in the subject. Effective amounts may vary, as recognized by those skilled in the art, depending on route of administration, excipient usage, and the possibility of co-usage with other agents. The term “treating” refers to administering one or more of the above-described dihydrobenzoquinone compounds to a subject that has cancer, or has a symptom of cancer, or has a predisposition toward cancer, with the purpose to cure, heal, alleviate, relieve, alter, remedy, ameliorate, improve, or affect the cancer, the symptoms of the cancer, or the predisposition toward the cancer.

To practice this method, a dihydrobenzoquinone compound can be administered orally, parenterally, by inhalation spray, or via an implanted reservoir. The term “parenteral” as used herein includes subcutaneous, intracutaneous, intravenous, intramuscular, intraarticular, intraarterial, intrasynovial, intrasternal, intrathecal, intralesional and intracranial injection or infusion techniques.

An oral composition can be any orally acceptable dosage from including, but not limited to, tablets, capsules, emulsions and aqueous suspensions, dispersions and solutions. Commonly used carriers for tablets include lactose and corn starch. Lubricating agents, such as magnesium stearate, are also typically added to tablets. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

A sterile injectable composition (e.g., aqueous or aleoaginous suspension) can be formulated according to techniques known in the art using suitable dispersing or wetting agents (such as, for example, Tween 80) and suspending agents. The sterile injectable preparation can also be a sterile injectable solution or suspension in a non-toxic parenterally acceptable diluent or solvent, for example, as a solution in 1,3-butanediol. Among the acceptable vehicles and solvents that can be employed are mannitol, water, Ringer's solution and isotonic sodium chloride solution. In addition, sterile, fixed oils are conventionally employed as a solvent or suspending medium (e.g., synthetic mono- or diglycerides). Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions can also contain a long-chain alcohol diluent or dispersant, or carboxymethyl cellulose or similar dispersing agents.

An inhalation composition can be prepared according to techniques well known in the art of pharmaceutical formulation and can be prepared as solutions in saline, employing benzyl alcohol or other suitable preservatives, absorption promoters to enhance bioavailability, fluorocarbons, and/or other solubilizing or dispersing agents known in the art.

A topical composition can be formulated in form of oil, cream, lotion, ointment and the like. Suitable carriers for the composition include vegetable or mineral oils, white petrolatum (white soft paraffin), branched chain fats or oils, animal fats and high molecular weight alcohols (greater than C12). The preferred carriers are those in which the active ingredient is soluble. Emulsifiers, stabilizers, humectants and antioxidants may also be included as well as agents imparting color or fragrance, if desired. Additionally, transdermal penetration enhancers can be found in U.S. Pat. Nos. 3,989,816 and 4,444,762. Creams are preferably formulated from a mixture of mineral oil, self-emulsifying beeswax and water in which mixture the active ingredient, dissolved in a small amount of an oil, such as almond oil, is admixed. An example of such a cream is one which includes about 40 parts water, about 20 parts beeswax, about 40 parts mineral oil and about 1 part almond oil. Ointments may be formulated by mixing a solution of the active ingredient in a vegetable oil, such as almond oil, with warm soft paraffin and allowing the mixture to cool. An example of such an ointment is one which includes about 30% almond and about 70% white soft paraffin by weight.

A carrier in a pharmaceutical composition must be “acceptable” in the sense that it is compatible with active ingredients of the formulation (and preferably, capable of stabilizing it) and not deleterious to the subject to be treated. For example, solubilizing agents, such as cyclodextrins (which form specific, more soluble complexes with one or more of active compounds of the extract), can be utilized as pharmaceutical excipients for delivery of the active ingredients. Examples of other carriers include colloidal silicon dioxide, magnesium stearate, cellulose, sodium lauryl sulfate, and D&C Yellow #10.

Further, the dihydrobenzoquinone compounds of this invention can be co-administered with an effective amount of one or more chemotherapeutic agents such as cisplatin, paclitaxel, podophyllotoxin, vincristin, plicamycin, dactinomycin, adriamycin, and 5-fluorouracil. The term “co-administering” refers to administering to a subject two or more active agents at the same time or at different time during cancer treatment.

In addition, a subject to be treated or treated with a dihydrobenzoquinone compounds can be co-treated with radiation. The radiation can be applied before, during, or after administration of the dihydrobenzoquinone compound. It can be ionizing radiation or non-ionizing radiation. Ionizing radiation has sufficient energy to interact with an atom and remove electrons from their orbits, causing the atom to become charged or “ionized.” It includes radiation with gamma ray, X-ray, neutrons, electrons, alpha particles, and beta particles. Non-ionizing radiation is electromagnetic radiation that does not have sufficient energy to remove electrons from their orbits. It includes radiation with ultraviolet rays, visible light, infrared light, microwave, and radio waves.

Suitable in vitro assays can be used to preliminarily evaluate the efficacy of the above-described dihydrobenzoquinone compounds in inhibiting proliferation of cancer cells. The compounds can further be examined for its efficacy in treating cancer by in vivo assays. For example, the compounds can be administered to an animal (e.g., a mouse model) having cancer and its therapeutic effects are then accessed. Based on the results, an appropriate dosage range and administration route can also be determined. In a similar manner, the in vitro and in vivo assays can also be used to evaluate efficacy of the compound in the presence of a chemotherapeutic agent or radiation.

Without further elaboration, it is believed that the above description has adequately enabled the present invention. The following specific examples are, therefore, to be construed as merely illustrative, and not limitative of the remainder of the disclosure in any way whatsoever. All of the publications and U.S. patent application Ser. No. 11/156,210 cited herein are hereby incorporated by reference in their entirety.

Chemical Syntheses:

### EXAMPLE 1

0.37 g of a powder containing IqA and IqB (Shandong Xinhua pharmaceutical Company, Shandong, China) at the ration of 85:15 were dissolved in 15 ml of analytical grade alcohol. To this solution were added 10 ml of an aqueous solution containing 1.5 mmol of glutathione (GSH). The resulting solution was stirred at room temperature for 60 minutes and then filtered to collect pale yellow precipitate to provide a crude product, i.e., Mixture A. Four isomers, i.e., A₁, A₂, A₃, and A₄ (structures shown below), were purified from the crude product by silica gel chromatography. The total yield of the four isomers was 84%, and A₁, A₂, A₃, and A₄ were at the ration of 2:1:0.2:0.1.

ESIMS (m/z) for the raw product: 691, 693:

¹HNMR(DMSO-d6) for A₁: 6.50 (s, 1 H), 5.36 (m, 2 H), 4.85 (dd, J=3.7, 8.5 Hz, 1 H), 4.14 (s, 2 H), 3.71 (s, 3 H), 3.49 (t, 1 H), 3.35 (s, 2 H), 3.18 (dd, J=13.7, 3.6 Hz, 1 H), 2.93 (dd, J=8.5, 13.6 Hz, 1 H), 2.86 (t, J=8 Hz, 2 H), 2.18 (m, 2 H), 2.06 (m, 2 H), 1.2-2.0 (m, 26 H), 0.88 (t, J=7.2 Hz, 3 H).

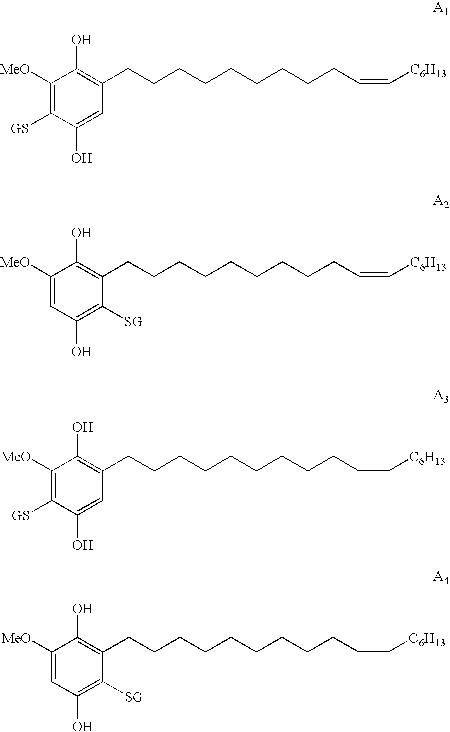

### EXAMPLE 2

Mixture B was prepared according to the procedure described in Example 1 except that methyl 2-thiohydroxy-acetate was used instead of GSH. It contained B₁, B₂, B₃, and B₄ (structures shown below) at the ratio of 2:1:0.2:0.1. The total yield was 90%. ¹H NMR (CDCl₃) for B₁:6.46 (s, 1 H), 5.36 (m, 2 H), 5.27 (s, 1 H), 3.85 (s, 3 H), 3.71 (s, 3 H), 3.35 (s, 2 H), 2.86 (t, J=8 Hz, 2 H), 1.2-2.0 (m, 26 H), 0.88 (t, J=7.2 Hz, 3 H).

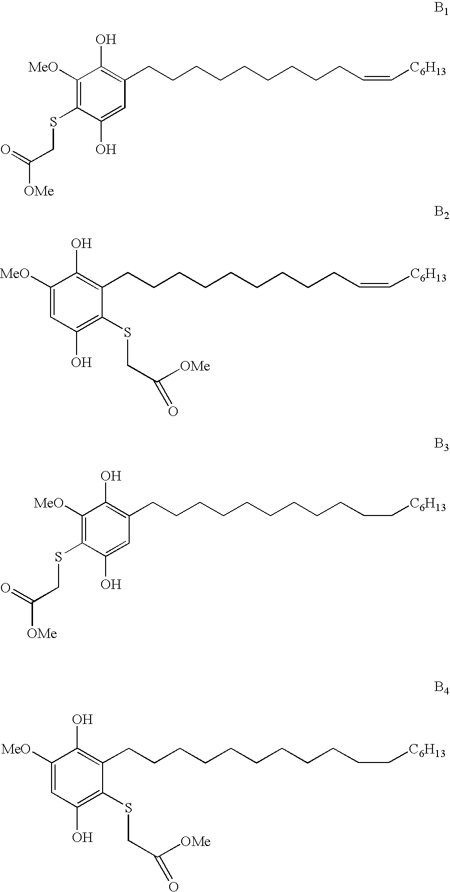

### EXAMPLE 3

Mixture C was prepared according to the procedure described in Example 1 except that cystein was used instead of GSH. It contained C₁, C₂, C₃, and C₄ (structures shown below) at the ratio of 2:1:0.2:0.1. The total yield was 75%.

ESIMS (m/z) for the mixture: 509, 511;

¹HNMR(DMSO-d6) for C₁: 6.50 (s, 1 H), 5.36 (m, 2 H), 4.65 (dd, J=3.7, 8.4 Hz, 1 H), 3.71 (s, 3 H), 3.35 (s, 2 H), 3.28 (dd, J=13.7, 3.6 Hz, 1 H), 2.98 (dd, J=13.6, 8.4 Hz, 1 H), 2.86 (t, J=8 Hz, 2 H), 1.2-2.0 (m, 26 H), 0.88 (t, J=7.2 Hz, 3 H). 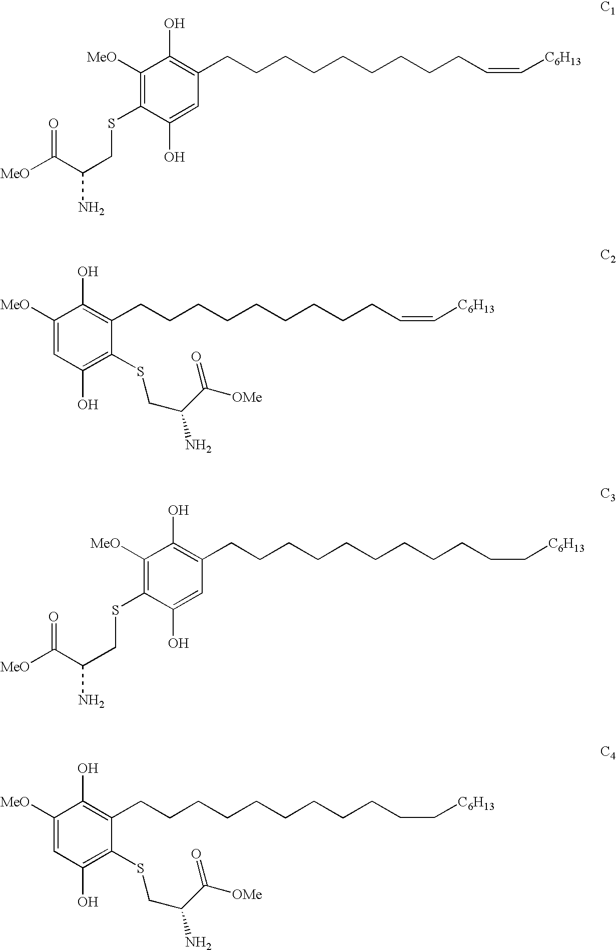
Biological Assay

In vitro assays were conducted to evaluate the efficacy of the above-obtained mixtures in inhibiting proliferation of cancer cells.

Three human tumor cell lines, i.e., Eca-109 (esophagus carcinoma cell line), BGC-823 (gastric adenocarcinoma cell line), and SPC-A1 (lung cancer cell line), were purchased from the Chinese Academy of Sciences and cultured in Iscove's Modified Dulbecco's Medium (IMDM) containing 10% fetal bovine serum (FBS) in an incubator at 37° C. under 5% CO₂. Cells at 70˜80% confluence were trypsinized, resuspended in IMDM medium containing 10% FBS at 1×10⁵ cells/ml, and seeded in 96-well plates (100 μl in each well). The plates were incubated at 37° C. under 5% CO₂ overnight.

Mixture A was dissolved in dimethyl sulfoxide (DMSO) to prepare a 10 mg/ml solution. The solution was diluted with growth medium and was subsequently added to wells containing cancer cells. The final concentrations of Mixture A in the wells were 100, 30, 10, 3, 1, and 0.3 μg/ml. Wells containing cancer cells and 10 μl of DMSO, but no Mixture A, were used as controls. Wells that contained cancer cells, but not DMSO and Mixture A were used as the background. The plates were then incubated at 37° C. under 5% CO₂ for 48 hrs.

To each of the wells, except for the background wells, were added 10 μl aliquots of 5 mg/ml 3-(4,5-dimethylthiazol-2-yl)-2,5-diphenyltetrazolium bromide. After being incubated for additional 3-4 hrs, the plates were spun at 1000 rpm for 15 minutes and the supernatants were carefully removed by vacuum. The cells were washed with 150 μl of phosphate-buffered saline.

After addition of 150 μl of DMSO to each well, the plates were placed on a shaker and spun at 150 rpm for 15 minutes to dissolve the precipitate in the wells. Absorbance was measured at 492 nm using a microplate reader. All assays were conducted in triplicate.

A software program, XLfit (ID Business Solutions), was used to calculate the concentration required to reach 50% inhibition (i.e., IC₅₀) on each cancer cell line.

Mixtures B and C, IqA (Shandong Xinhua Pharmaceutical Co. Ltd, China), and cisplatin (Qilu Pharmaceutical Ltd., China) were each tested against Eca-109, BGC-823, and SPC-A1 in the manner similar to that described above. Their IC50 values were calculated using XL fit.

The results show that Mixtures A, B, and C suprisingly inhibited the proliferation of all three cancer cell lines. Even more surprisingly, Mixtures B and C were more effective than IqA in inhibiting the proliferation of the cancer cell lines.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above described, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. A compound of formula (I) 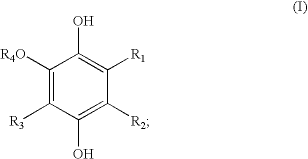
wherein
R₁ is C₁₋₂₀ alkyl, C₂₋₂₀ alkenyl, C₂₋₂₀ alkynyl, or aryl;
one of R₂ and R₃ is XRₐ and the other of R₂ and R₃ is H, C₁₋₆ alkyl, C₂₋₆ alkenyl, C₂₋₆ alkynyl, C₃₋₁₂ cycloalkyl, C₁₋₁₂ heterocycloalkyl, aryl, heteroaryl, or YR_{b}; in which each of X and Y, independently, is O, S, Se, or NR′, and each of Rₐ and R_{b}, independently, is H, C₁₋₆ alkyl, C₂₋₆ alkenyl, C₂₋₆ alkynyl, C₃₋₁₂ cycloalkyl, C₁₋₁₂ heterocycloalkyl, aryl, heteroaryl, C(O)R′, S(O)₂R′, an amino acid moiety, or an oligopeptide moiety; R′ being H, C₁₋₆ alkyl, C₂₋₆ alkenyl, C₂₋₆ alkynyl, C₃₋₁₂ cycloalkyl, C₁₋₁₂ heterocycloalkyl, aryl, or heteroaryl; and
R₄ is H, C₁₋₆ alkyl, C₂₋₆ alkenyl, C₂₋₆ alkynyl, C₃₋₁₂ cycloalkyl, C₁₋₁₂ heterocycloalkyl, aryl, or heteroaryl.

2. The compound of claim 1, wherein R₁ is (CH₂)₉CH═CH(CH₂)₅CH₃ or (CH₂)₁₆CH₃.

3. The compound of claim 2, wherein R₄ is methyl.

4. The compound of claim 3, wherein the other of R₂ and R₃ is H.

5. The compound of claim 4, wherein X is S.

6. The compound of claim 5, wherein Rₐ is 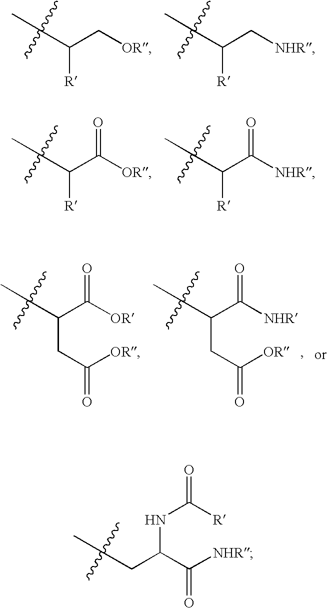
wherein each of R′ and R″, independently, is H, C₁₋₆ alkyl, C₂₋₆ alkenyl, C₂₋₆ alkynyl, C₃₋₁₂ cycloalkyl, C₁₋₁₂ heterocycloalkyl, aryl, or heteroaryl.

7. The compound of claim 5, wherein Rₐ is 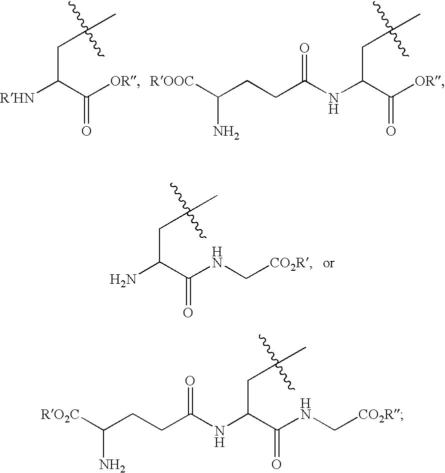
wherein each of R′ and R″, independently, is H, C₁₋₆ alkyl, C₂₋₆ alkenyl, C₂₋₆ alkynyl, C₃₋₁₂ cycloalkyl, C₁₋₁₂ heterocycloalkyl, aryl, or heteroaryl.

8. The compound of claim 1, wherein the other of R₂ and R₃ is H.

9. The compound of claim 8, wherein X is S.

10. The compound of claim 9, wherein Rₐ is 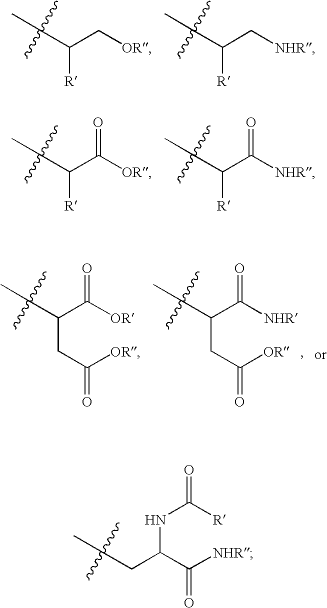
wherein each of R′ and R″, independently, is H, C₁₋₆ alkyl, C₂₋₆ alkenyl, C₂₋₆ alkynyl, C₃₋₁₂ cycloalkyl, C₁₋₁₂ heterocycloalkyl, aryl, or heteroaryl.

11. The compound of claim 9, wherein Rₐ is 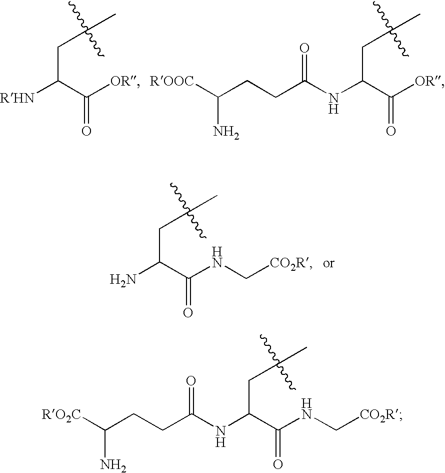
wherein each of R′ and R″, independently, is H, C₁₋₆ alkyl, C₂₋₆ alkenyl, C₂₋₆ alkynyl, C₃₋₁₂ cycloalkyl, C₁₋₁₂ heterocycloalkyl, aryl, or heteroaryl.

12. The compound of claim 1, wherein R₄ is methyl.

13. The compound of claim 1, wherein the compound is

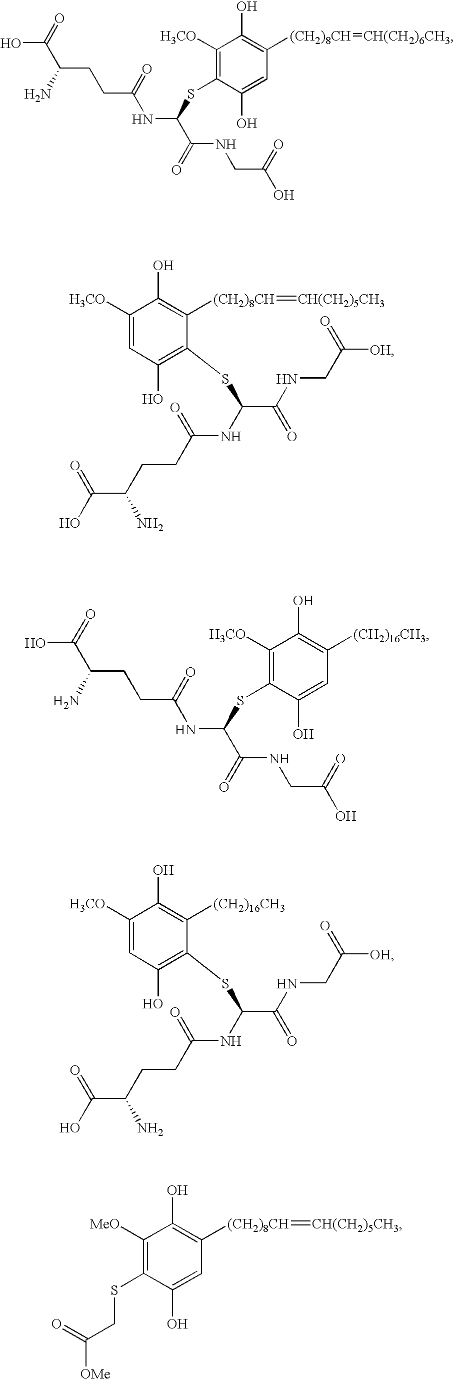

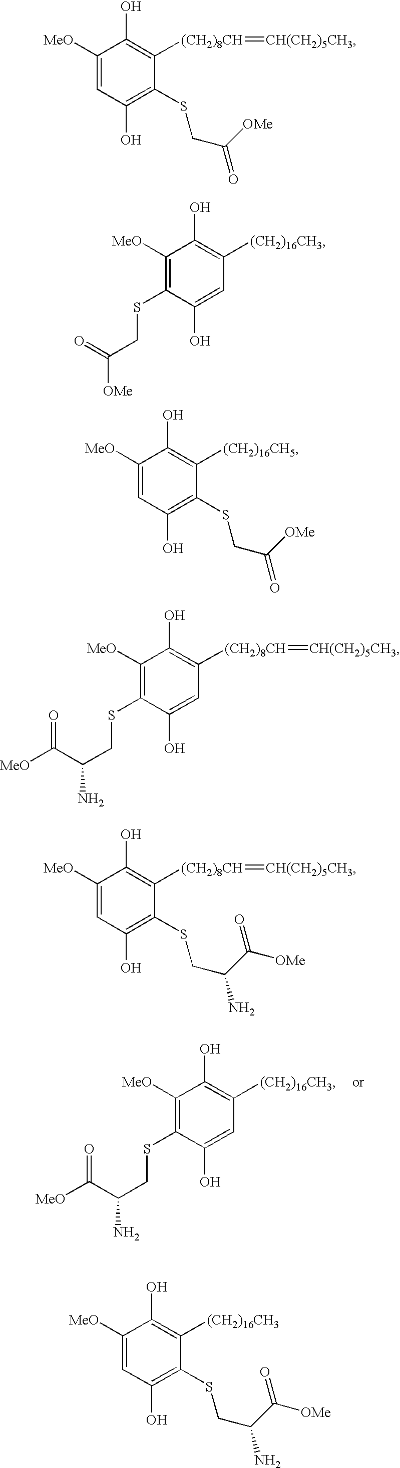

14. The compound of claim 1, wherein the compound is

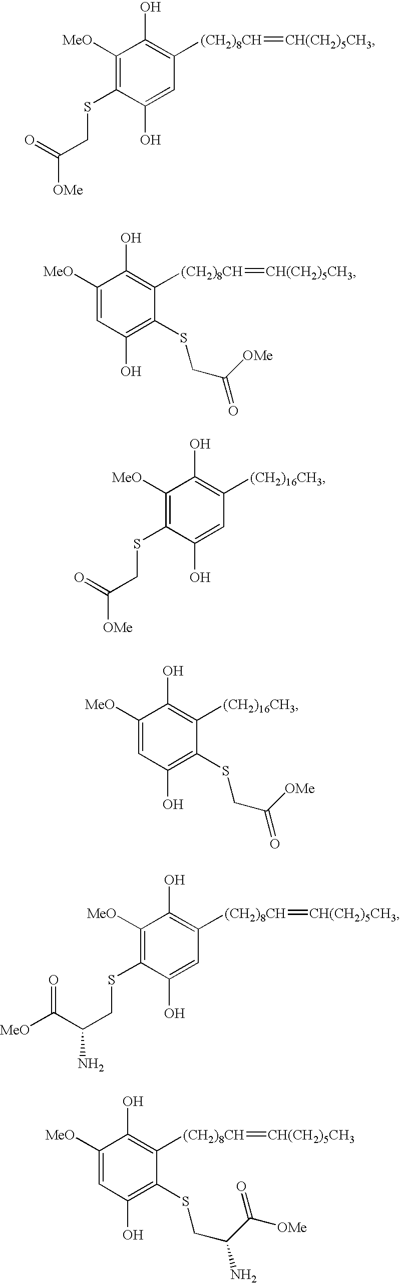

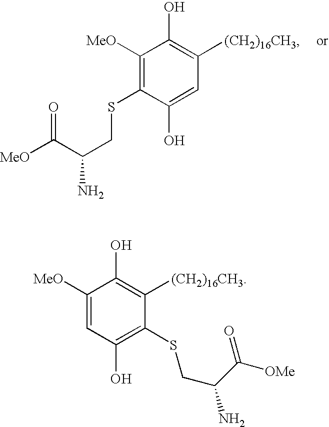

15. A process for preparing a compound of formula (II): 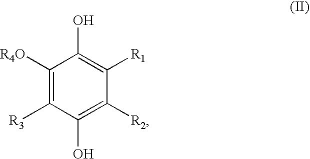
wherein R₁ is C₁₋₂₀ alkyl, C₂₋₂₀ alkenyl, C₂₋₂₀ alkynyl, or aryl; one of R₂ and R₃ is XRₐ and the other is H; in which X is O, S, Se, or NR′; and Rₐ is H, C₁₋₆ alkyl, C₂₋₆ alkenyl, C₂₋₆ alkynyl, C₃₋₁₂ cycloalkyl, C₁₋₁₂ heterocycloalkyl, aryl, heteroaryl, C(O)R′, S(O)₂R′, an amino acid moiety, or an oligopeptide moiety; R′ being H, C₁₋₆ alkyl, C₂₋₆ alkenyl, C₂₋₆ alkynyl, C₃₋₁₂ cycloalkyl, C₁₋₁₂ heterocycloalkyl, aryl, or heteroaryl; and R₄ is H, C₁₋₆ alkyl, C₂₋₆ alkenyl, C₂₋₆ alkynyl, C₃₋₁₂ cycloalkyl, C₁₋₁₂ heterocycloalkyl, aryl, or heteroaryl; said process comprising reacting HXRₐ, in which X and Rₐ are defined above, with a compound of formula (III): 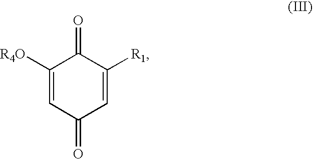
wherein R₁ and R₄ are defined above.

16. A method of treating cancer comprising administering to a subject in need thereof an effective amount of the compound of claim 1.

17. The method of claim 16, wherein the compound is

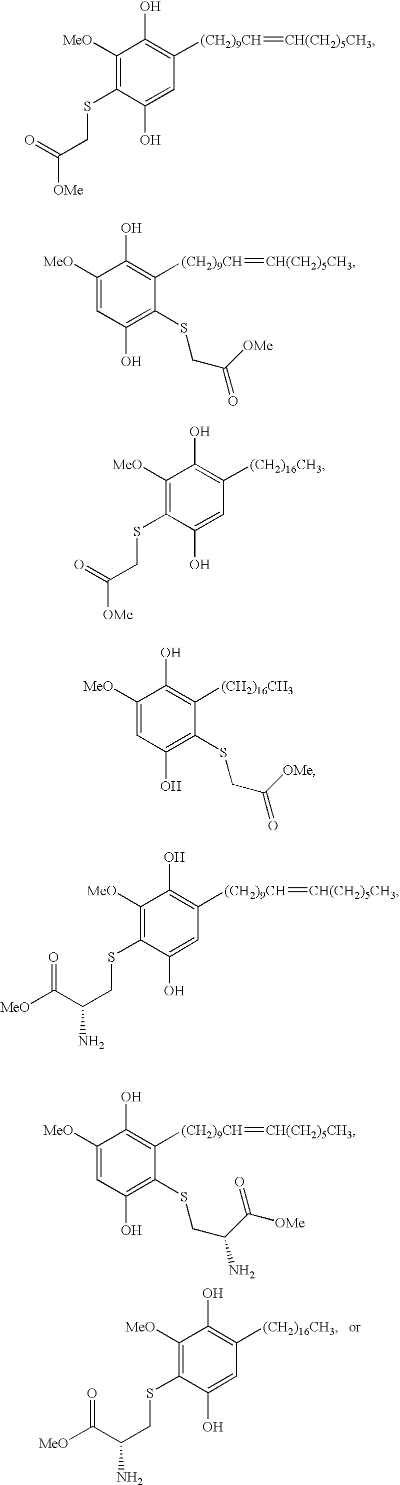

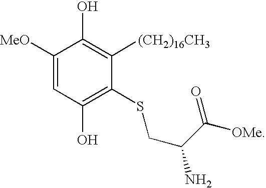

18. The method of claim 16, wherein the cancer is oesophagus carcinoma, head and neck carcinoma, gastric carcinoma, lung carcinoma, or colon carcinoma.

19. The method of claim 18, wherein the cancer is oesophagus carcinoma, gastric carcinoma, or lung carcinoma.

20. The method of claim 16, wherein the compound is co-administered to the subject with an effective amount of one or two additional chemotherapeutic agents.

21. The method of claim 20, wherein said one or two additional chemotherapeutic agents are selected from: cisplatin, mitomycin C, bleomycin, topotecan, irinotecan, gemcitabine, docetaxel, paclitaxel, podophyllotoxin, vincristin, plicamycin, daunorubicin, dactinomycin, adriamycin, 5-fluorouracil, hormones, hormone antagonists, and cytokines.

22. The method of claim 16, wherein the subject is co-treated with radiation therapy.

23. The method of claim 19, wherein the subject is co-treated with radiation therapy.
